(19) United States
(12) Patent Application Publication
(10) Pub. No.: US 2015/0283395 A1
(43) Pub. Date: Oct. 8, 2015

(54) REAL TIME MONITORING AND STIMULATION OF HUMAN BRAIN USING SUPERDIRECTIVE NEAR FIELD ARRAYS FOR FOCUSED TRANSCRANIAL MAGNETIC STIMULATION

(71) Applicant: WILKES UNIVERSITY, Wilkes-Barre, PA (US)

(72) Inventor: Abas SABOUNI, Kingston, PA (US)

(21) Appl. No.: 14/676,913

(22) Filed: Apr. 2, 2015

Related U.S. Application Data
(60) Provisional application No. 61/975,163, filed on Apr. 4, 2014.

(51) Int. Cl. A61N 2/00; A61N 2/02

(52) U.S. Cl. CPC A61N 2/006; A61N 2/02

(57) ABSTRACT

A superdirective near field array for transcranial magnetic stimulation includes a plurality of electromagnetic coils or elements arranged in a superdirective array. An excitation source induces an electrical current in one or more of the electromagnetic coils. A controller is programmed to direct or position the elements to generate a magnetic field in an excitation region or regions of a human head or brain. The array coils focus a predetermined current density in the excitation region or regions, and reposition the excitation region to various locations around the head or brain by selectively varying a phase and a magnitude of the electrical current in selected array elements. Also, a method includes providing electromagnetic coils in an array and an excitation source, generating a magnetic field in regions of a brain by focusing a current density in the excitation region, and varying the currents in the elements.

### CROSS-REFERENCE TO RELATED APPLICATIONS

This application claims priority to and the benefit of U.S. Provisional Application No. 61/975,163 filed Apr. 4, 2014, entitled “SUPERDIRECTIVE NEAR FIELD ARRAYS FOR FOCUSED TRANSCRANIAL MAGNETIC STIMULATION”, which is hereby incorporated by reference in its entirety.

### BACKGROUND

The application generally relates to a method and system for transcranial magnetic stimulation. The application relates more specifically to an automated method and system for transcranial magnetic simulation using superdirective near field arrays to focus magnetic fields and image the eddy current generated inside the brain due to magnetic field.

Transcranial Magnetic Stimulation (TMS) is a technique that uses intense pulsed magnetic fields to induce currents in neuronal tissues which produce therapeutic effects in the brain. Electromagnetic coils are placed adjacent to the cranium or scalp of a patient while high-intensity current is induced in the coils. If the induced currents are large enough, neurons may be locally depolarized. By localizing the magnetic field with prior anatomical MRI information, it is possible to modulate cortical function by exciting or inhibiting neuronal activity in a local area. TMS may be used to treat neurological disorders such as depression. TMS may also be used to measure the connection between the primary motor cortex and a muscle, to evaluate damage from spinal cord injuries. TMS techniques are also being considered for treatment of a broad range of other neurological problems such as stroke, Parkinson's disease, and schizophrenia.

In the existing TMS techniques in use today, magnetic fields are applied to the brain using two large electromagnetic coils. The magnetic field is spread across large areas of the brain. However, the present TMS approach has several disadvantages. First, TMS techniques are unable to pinpoint a specific structure in the brain that is smaller than the excitation region of the coils, e.g., approximately ten cubic centimeters (10 cm³). Second, even with the relatively large excitation region, it is possible that the magnetic field generated by the TMS technique may miss the appropriate region of the brain, because the appropriate region can vary from patient to patient.

The majority of research on TMS techniques is directed to the efficacy of the technique. Most of the electromagnetic research in this area is on development of simulation approaches. Few researchers have investigated the design of new source types for TMS techniques. Those papers that disclose new source designs include larger arrays of existing coils, genetic algorithms for optimizing existing arrays, conductive shielding approaches, and simulations of applying metamaterials. However, these designs of sources or arrays for TMS have numerous drawbacks and are far from optimal.

Intended advantages of the disclosed systems and/or methods satisfy one or more of these needs or provide other advantageous features. Other features and advantages will be made apparent from the present specification. The teachings disclosed extend to those embodiments that fall within the scope of the claims, regardless of whether they accomplish one or more of the aforementioned needs.

### SUMMARY

One embodiment relates to a superdirective near field array for transcranial magnetic stimulation comprising a plurality of electromagnetic coils or elements arranged in a superdirective array, an excitation source for inducing an electrical current in one or more of the electromagnetic coils, and a controller programmed to position or direct the elements to generate a magnetic field in at least one excitation region of a living organism, e.g., a human head or brain; the array coils are configured to focus a predetermined current density in the excitation region or regions using near-field sub-wavelength focusing, and to reposition the excitation region to various locations around the head or brain by varying a phase and a magnitude of the electrical current in predetermined array elements.

Another embodiment relates to a method for transcranial magnetic stimulation comprising providing a plurality of electromagnetic coils or elements arranged in a superdirective array, an excitation source for inducing an electrical current in one or more of the electromagnetic coils, and a controller programmed to generate a magnetic field; generating a magnetic field using near-field sub-wavelength focusing in at least one excitation region of a human head or brain by focusing a predetermined current density in the excitation region or regions; varying a phase and a magnitude of the electrical current in predetermined array elements; and repositioning the excitation region to one or more locations around the head or brain.

Advantages of the embodiments described herein include a new TMS coil system that precisely focuses electromagnetic fields inside the human brain. Further, the disclosed TMS coil system is capable of electronically scanning the magnetic field region within the brain.

In addition, the disclosed TMS coil system supports various waveforms that may be applied simultaneously to different regions of the brain, e.g., high frequency pulses to enhance activity in one region and simultaneous low frequency pulses to suppress activity in another.

Alternative exemplary embodiments relate to other features and combinations of features as may be generally recited in the claims.

### BRIEF DESCRIPTION OF THE FIGURES

FIG. 1 is an exemplary embodiment of a TMS coil array for generating a localized magnetic field in a human brain.

FIG. 2 is a plot of the current density inside a simulated human brain for existing 4.5 cm figure-8 coils.

FIG. 3 shows a plot of the current density inside a simulated human brain for a simple 10-element linear array of 3 mm coils in accordance with an exemplary embodiment.

FIG. 4 shows a broad magnetic field created in the brain by phasing all coils together in accordance with an exemplary embodiment.

FIG. 5 shows two different regions of the brain excited together in accordance with an exemplary embodiment.

FIG. 6 shows a block diagram of the automated TMS system.

FIG. 7a shows MRI data in the sagittal plane of a patient.

FIG. 7b shows segmented MRI data in the sagittal plane of a patient.

FIG. 7c shows a map of conductivity in the sagittal plane of a patient.

FIG. 7d shows a map of conductivity in the coronal plane of a patient.

FIG. 7e shows a gradient color scale for identifying components of the images in FIGS. 7a-7d.

FIG. 8 shows a block diagram of the implementation of FEM code.

FIGS. 9a and 9b show a map of induced current for circular coil at C position in FIG. 10. FIG. 9b shoes the map of induce current and FIG. 9a shows induce current mapped on the MRI in the sagittal plane.

FIG. 10 shows a circular coil placed at the top of the head.

FIG. 11 shows five different locations (A, B, C, D and E) for placement of the circular coil of FIG. 10.

FIGS. 12a and b show a map of induced current for circular coil at position A in FIG. 11. FIG. 12a shows the map of induced current and FIG. 12b shows induce current mapped on the MRI (sagittal plane).

FIGS. 13a and b show a map of induced current for circular coil at B position in FIG. 11. FIG. 13a shows the map of induced current and FIG. 13b shows induce current mapped on the MRI (sagittal plane).

FIGS. 14a and b show a map of induced current for circular coil at C position in FIG. 11. FIG. 14a shows the map of induced current and FIG. 14b shows induce current mapped on the MRI (sagittal plane).

FIGS. 15a and b show a map of induced current for circular coil at D position in FIG. 11. FIG. 15a shows the map of induced current and FIG. 15b shows induce current mapped on the MRI (sagittal plane).

FIGS. 16a and b show a map of induced current for circular coil at E position in FIG. 11. FIG. 16a shows the map of induced current and FIG. 16b shows induce current mapped on the MRI (sagittal plane).

### DETAILED DESCRIPTION OF THE EXEMPLARY EMBODIMENTS

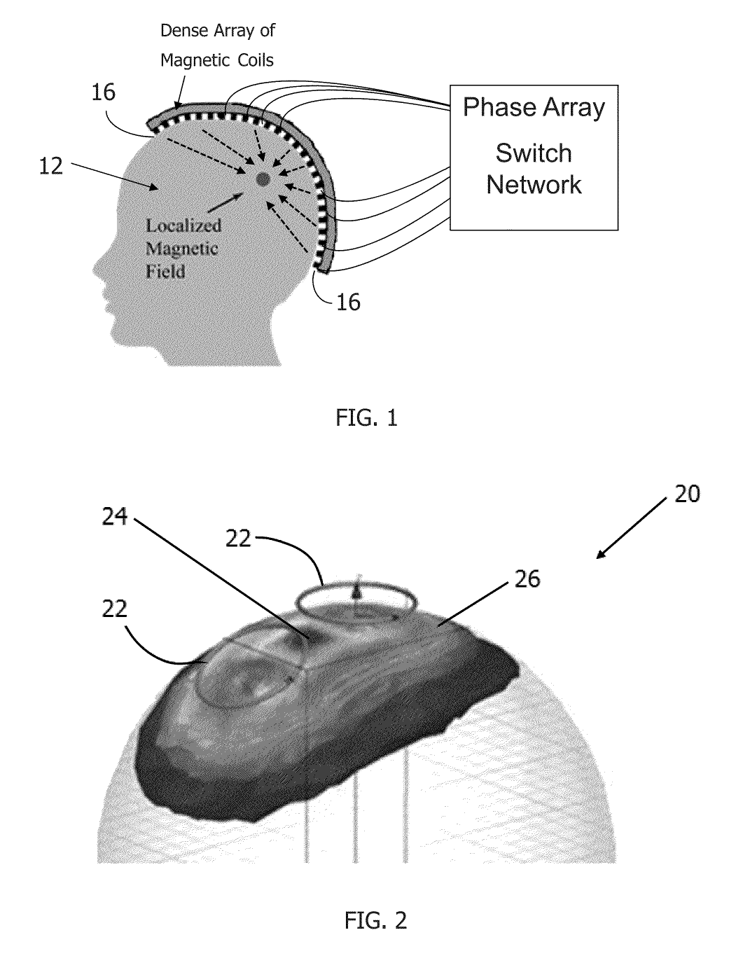

Referring to FIG. 1, a focused TMS coil array 10 combines features such as sub-diffraction limit focusing, and superdirective arrays, to provide a system for focusing electromagnetic fields within a living organism, e.g., the human body, to much smaller dimensions than that which is currently possible in the prior art. Using a dense array of electromagnetic elements 16, it is possible to produce highly localized magnetic fields within the brain 12, and to electronically scan across various brain structures. In fact, a dense phased array of magnetic coils (or TMS) 10 as illustrated in FIG. 1 provides a precise excitation region that is electronically scanned across the brain 12. TMS array coil 10 focuses a region 14 of maximum current density in a few cubic millimeters. Region 14 may be repositioned to various locations around the head by varying the phase and magnitude of the electrical current in different electromagnetic array elements 16. Current TMS systems involve a pair of coils which excite a single spot several cm in diameter, about the size of a golf ball. The dense array provides an excitation area approximately 25 times smaller, or 1/25^{th} of, the minimum excitation area achieved with current TMS systems. In one embodiment resolution of the excitation area is on the order of 5 mm, with pixels that can be electronically patterned over the entire surface of the brain.

The current generated in each array element 16 may be up to 100 times greater than the current generated in existing figure-8, or double coil probes. Elements 16 produce about the same current density in the brain 12, although in a much more focused area of the brain 12. Since array elements 16 are positioned very near to adjacent array elements 16, strong electromagnetic coupling occurs between array elements 16. Coupling may cause difficulty in applying the necessary phase relationships for scanning E.g., high currents and electromagnetic coupling together will manifest as an impedance matching problem, which may vary with frequency and scan conditions.

In one embodiment, TMS array coil 10 may be arranged as a superdirective array. A superdirective array is an array that has higher directivity than is suggested by the diffraction limit of the array elements 16, by using a very dense lattice of array elements 16 arranged to produce nulls at locations that are not part of the narrow main beam. This usually results in high current magnitudes, and currents in neighboring elements that are nearly 180 degrees out of phase. To achieve the required 100 microsecond (μs) rise time for TMS, the coil inductance must be scaled down by a factor of 10, to a maximum of 30 turns. Such a coil 10 would have a resistance of 4 Ohms (Ω) without changing the wire diameter, or 0.4Ω if the coiled wires are 10 times larger, keeping the overall coil 10 volume the same. In one exemplary embodiment the coil 10 requires 173 amperes (A) to generate 1 Tesla (T) magnetic fields, dissipating 12 kilowatts (kW). In the exemplary embodiment pulses only last for about 200 μs. Thus, the total power dissipated equals 2.4 joules (J)/pulse. At a pulse rate of 10 Hz, average power dissipation is approximately 24 Watts (W) in each coil 10.

If the coil diameter is 1 cm, a hemispherical array covering half the average human head having a diameter of 17.5 cm would require roughly 1,400 coils, and consume 33 kW if the entire array were driven simultaneously. In actual use, perhaps 5% of the array would be driven at one time and the active region would be scanned across the brain. Such a design would consume 1.6 kW, or about as much as a hair dryer, so cooling still needs to be considered in the design

The diffraction limit which proscribes the visualization of features smaller than about half of the wavelength, is only valid in the far field zone:

(r>2d²/λ)

where d is the greater size of the phase array coil 10 and r and λ are the distance from the object and wavelength, respectively. In one embodiment the phase array coil 10 may be located very close to the object therefore it can be considered in the near field region. In a near field zone the evanescent components contain fine detail of the electromagnetic field distribution which allows the user to see beyond the diffraction limit. A substantial fraction of the emitted electromagnetic energy can be sent into an arbitrarily small solid angle.

By directivity what is meant is how ‘directional’ an antenna's radiation pattern is. The design of the dense phased coil array is based on the sub-diffraction limit focusing and the superdirective arrays, which provides a focused electromagnetic field to much smaller volume or area than is currently possible.

Such arrays may generate few electromagnetic coupling effects among array elements 16, and low radiation resistance. Low radiation resistance may make impedance matching difficult.

Electromagnetic coupling and low radiation resistance are controllable for the TMS array application. Signal/noise ratio and efficiency are not a concern, as may be the case in data transmission; the TMS array 10 operates at very low frequencies so that efficient matching circuits may be designed. Practical approaches for dealing with the low impedance of such TMS arrays at low frequencies include active matching networks, the use of magnetic material, and the use of the vertical dimension which has not yet been exploited in coil design.

In one embodiment, the disclosed method includes identifying phasing methods for an arbitrary set of sources to provide the smallest focal spot for currents excited in the brain. Superdirective array techniques are applied, and optionally, the superdirective TMS array 10 may be modified for use in a lossy material—i.e., brain material having dielectric conductivity greater than zero—and for near-field focusing. This aspect combines analytical electromagnetic calculations and simulations using the TMS numerical solver. In one embodiment, the numerical solver may be an analytical code base on the least square optimization technique with constrain condition capable of obtaining the amplitude and the phase of excitation to each coil. See, e.g., J. Fraleigh, R. Beauregard, and V. Katz, “Linear Algebra. World student series edition,” Addison-Wesley, vol. 53, p. 54, 1995. The analytical code allows the user to define the desired pattern of electric field in the tissue, and the pulse repetition pattern, and translates the electric field pattern and pulse repetition pattern into magnitude and direction of current in each of the coils. Software controls the coil array, e.g., LabView code operating on an embedded computer with digital I/O cards. The control software then triggers the pulses according to the desired waveform.

In another embodiment, the method discloses a TMS array 10 capable of achieving a predetermined current distribution e.g., the current distribution described above. TMS array 10 may include coils or other structures, magnetic materials, and impedance matching circuits. Other aspects of TMS array 10 may include shielding materials between array elements 16, and/or superconducting magnets to achieve the required magnetic field densities.

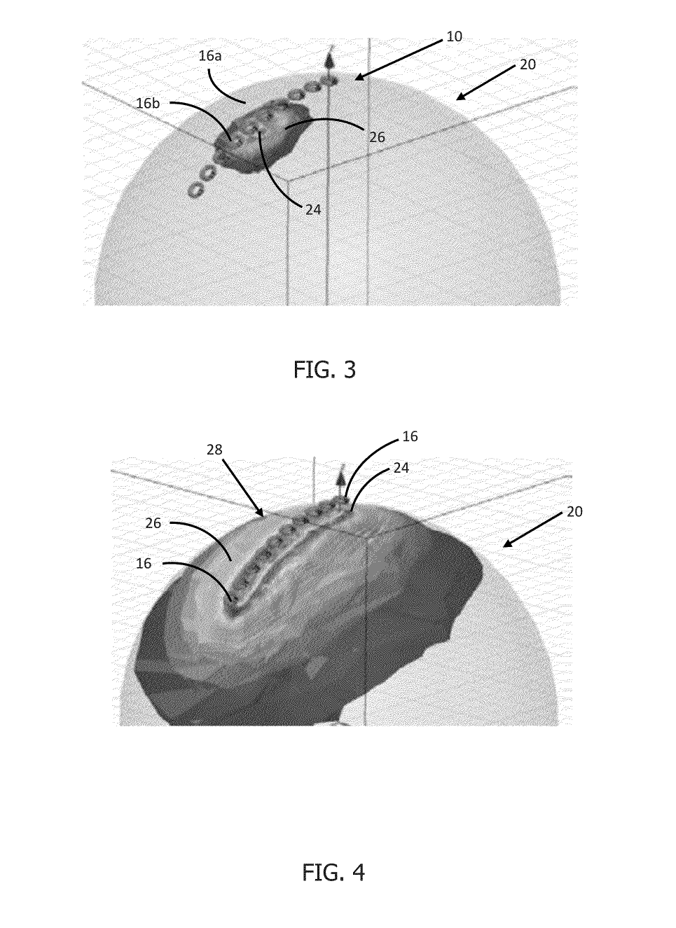

Referring next to FIGS. 2 and 3, a simplified model of a human brain 20 is illustrated. FIG. 2 shows a typical figure-8 coil 22 used in commercially available TMS devices. Plots of the current density inside a 20 cm diameter spherical volume of seawater, which is a close approximation to human tissue, are shown for existing 4.5 cm figure-8 coils (FIG. 2) and for a 10-element linear array of 3 mm coils (FIG. 3). The field is highest (red regions) in an area roughly equal to the coil size. Both arrays produce the same maximum current density. In FIG. 2, a first region 24 having the highest current density is indicated in red, and the magnetic field is about half of its maximum value at a second region 26 indicated in green. The current in both coils 22 is 180° out of phase, resulting in a maximum magnetic field and current density in between the coils 22, in region 24.

FIG. 3 shows an exemplary TMS array 10, wherein the TMS array 10 is a 10-element linear array. Two array elements 16a, 16b, of TMS array 10 are excited in the same way. The maximum field is the same, but the size of the focused region 24 of maximum current density is a few cubic millimeters, e.g., in one embodiment focused region 24 is on the order of 5 mm³. Focused region 24 can be moved about the head 20, or brain 12, by varying the phase and magnitude of the currents in different array elements 16.

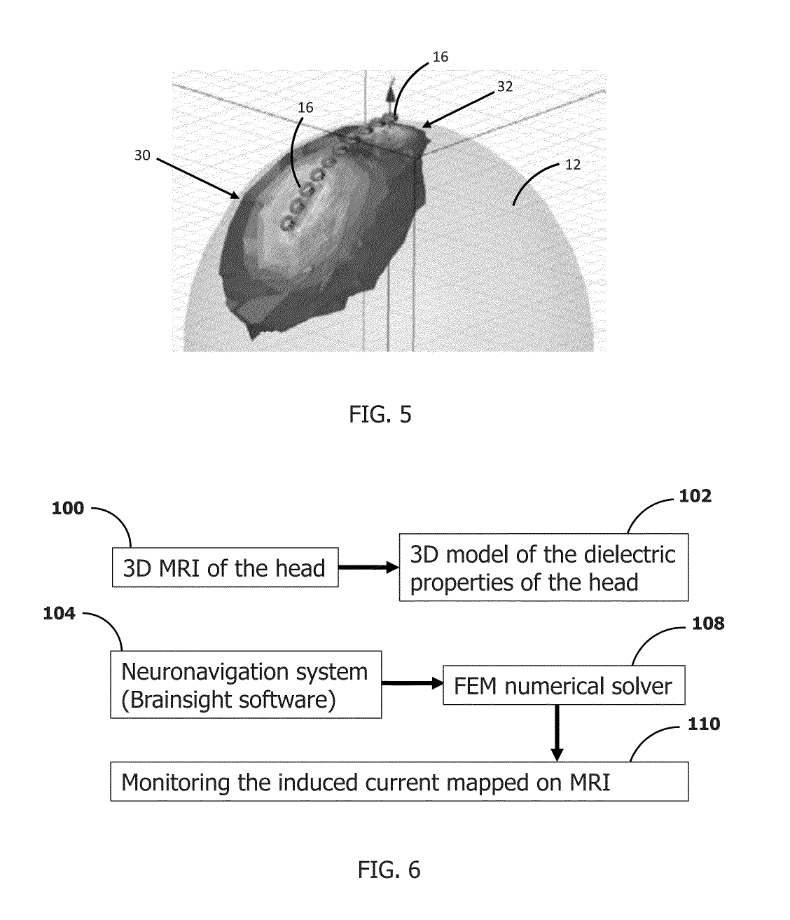

Referring to FIGS. 4 and 5, the size and shape of the excited region 24, 26 may be varied to excite broad regions 28 of the brain 12 (FIG. 4), or entirely separate regions 30, 32 of the brain 12 simultaneously, using different waveforms (FIG. 5). Using a two dimensional array, complex patterns of currents can be induced in the brain 12. A very dense array of elements 16 is used to achieve directivity in the far field that is greater than what is dictated by the diffraction limit. The superdirective array 10 is arranged for near-field sub-wavelength focusing. In an embodiment, array 10 may receive 1 KW to deliver only a few watts to the brain. The TMS array 10 may operate at very low frequencies. In one embodiment the TMS array 10 is operated in pulsed current mode. The bandwidth of the excitation signals spans from DC to the kHz or MHz range, where efficient matching circuits are easily designed. In another embodiment active circuits (not shown) provide broadband matching for dense arrays, which work well at low frequencies.

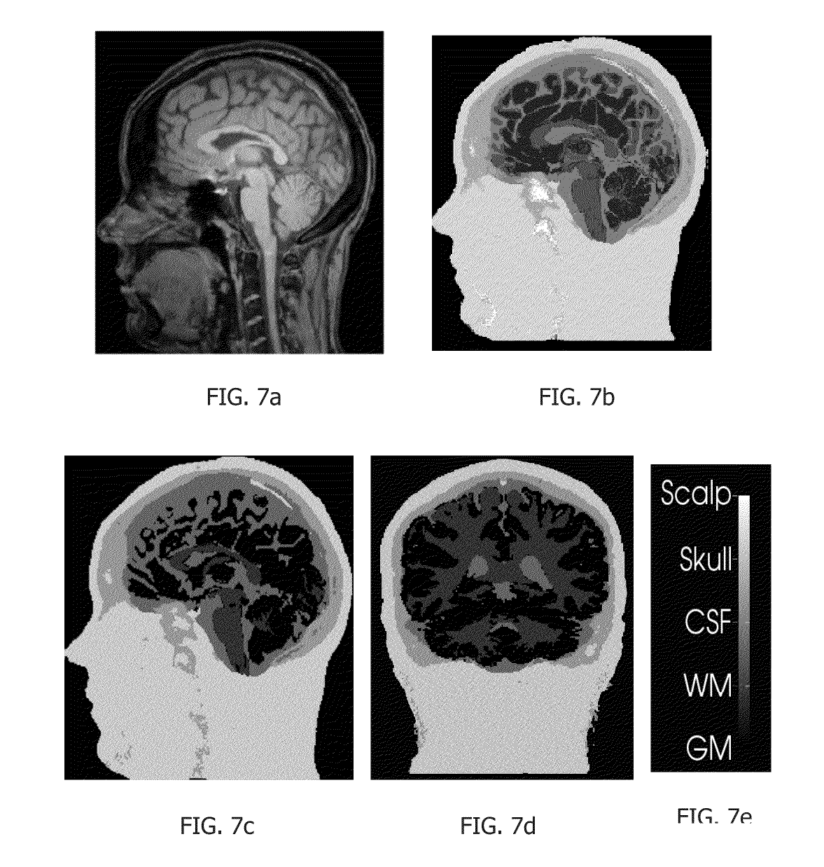

FIG. 6 shows a block diagram of the TMS system. To model induced current in a realistic brain, three dimensional (3D) Magnetic Resonance Imaging (MRI) of the brain (FIG. 7(a)) was acquired at step 100. MRI segmentation software may be used to categorize different regions of the brain such as Scalp, Skull, Cerebro-Spinal Fluid (CSF), Gray Matter (GM), and White Matter (WM) (FIG. 7(b)). This model may then be inserted as input to the TMS simulator and conductivity values assigned to each tissue type at step 102. FIGS. 7(c) and (d) show a cross-sectional view in sagittal and coronal planes of an exemplary segmented brain. The color bar 112 (FIG. 7(e)) indicates the different tissue type. Table I (below) shows the conductivity values for each brain tissue at 3.3 KHz frequency.

TABLE I

| Tissue | GM | WM | CSF | Skull | Scalp |
|---|---|---|---|---|---|
| σ(S/m) | 0.109 | 0.066 | 2.0 | 0.02 | 0.33 |

The output of the neuro-navigation system is also another input to the TMS simulator (FEM numerical solver) at step 104. A neuro-navigation system (e.g., Brainsight™) allows the TMS coil to be navigated and positioned over a specified target location based upon an individual MRI image. Based on information from the neuro-navigation system, the position and orientation of the coil is determined. The position and orientation of the coil may then be input to the TMS simulator to compute the expected flow of electric current in the brain at step 106. Results are then mapped on MRI of the patient at step 110. The TMS simulator is explained below.

### TMS Simulator

The frequency range for TMS is from DC to 10 kHz. In this range, the electromagnetic phenomenon satisfies Maxwell equations under quasi-static conditions.

$$∇ \times ( \frac{1}{σ} ∇ \times \vec{T} ) + j ω {μ}_{0} \vec{T} = - j ω {μ}_{0} {\vec{H}}_{s} + j ω {μ}_{0} ∇ Ω \qquad ( 1 )$$

The so-called T-Ω formulation, or current vector potential-magnetic scalar potential method, may be used to model the electromagnetic propagation:

Where:

j is an imaginary versor;

ω is pulsation;

T is the electric vector potential due to unknown currents in the head;

Ω (magnetic scalar potential) represents field induced in the brain tissues;

Hₛ, is an external magnetic field (TMS coil);

μₒ is the permeability of free space; and

σ is electric conductivity of the brain tissues.

In equation (1), if T is known then the eddy currents in the brain can be calculated as:

∇×\vec{T}=\vec{J}_{ind} (2)

where Jᵢₙd is the induced current in the brain. Electromagnetic properties of the human head are considered to be low conducting ones. Therefore the term ∇Ω can be neglected in equation (1). The generated external field, Hₛ can be calculated everywhere outside the source using the Biot-Savart Law. The partial differential equation (1) may be solved by a vector Finite Element Method (FEM) capable of considering arbitrary complex geometries such as anatomical structures in the human brain.

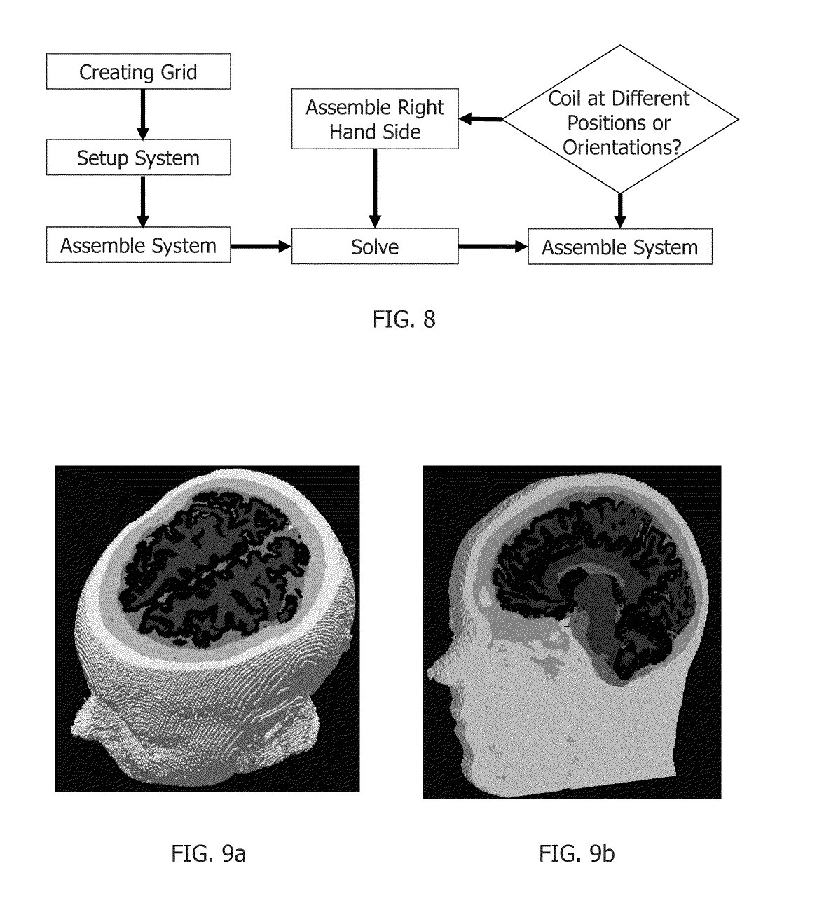

In an alternate embodiment, instead of working with complex valued finite elements directly, complex valued function T is split into its real part (Tᵣ) and imaginary (7) part. Separate scalar finite element fields are used for discretizing each one of them, as shown in equations (3), (4) and (5) below:

$$\begin{matrix}\vec{T} = {T}_{r} + j {T}_{j} & ( 3 ) \\ ∇ \times ( \frac{1}{σ} ∇ \times {T}_{r} ) - ω {μ}_{0} {T}_{j} = ω {μ}_{0} {H}_{sj} & ( 4 ) \\ ∇ \times ( \frac{1}{σ} ∇ \times {T}_{j} ) + ω {μ}_{0} {T}_{r} = - ω {μ}_{0} {H}_{sr} & ( 5 )\end{matrix}$$

where Hₛᵣ, and Hₛⱼ are the real and imaginary part of the external magnetic field, respectively. FIG. 8 shows an exemplary block diagram of the implementation of FEM code for solving equation (4) and equation (5) individually. An object-oriented scientific library (Deal.ii) includes a feature called dimension independent programming using C++ templates on locally adapted meshes, which make the TMS solver more efficient programming. In one embodiment parallel computing with Message Passing Interface (MPI) may be used in order to decrease the TMS solver run-time.

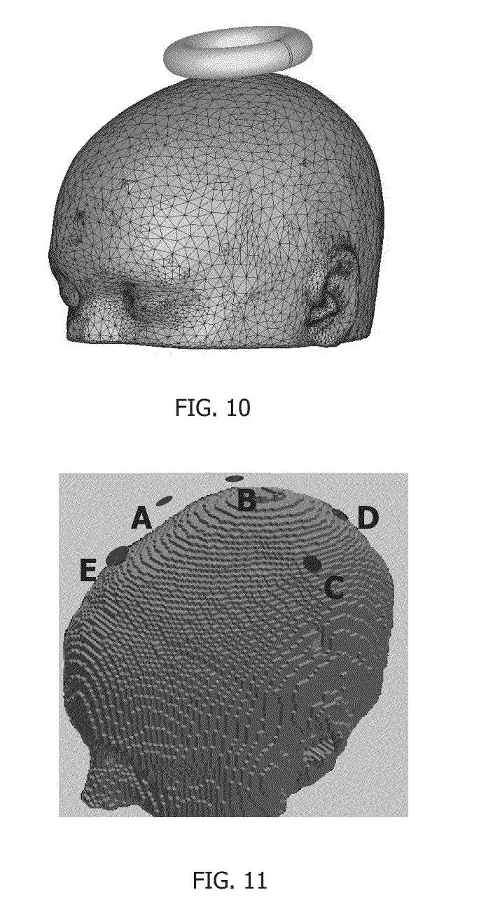

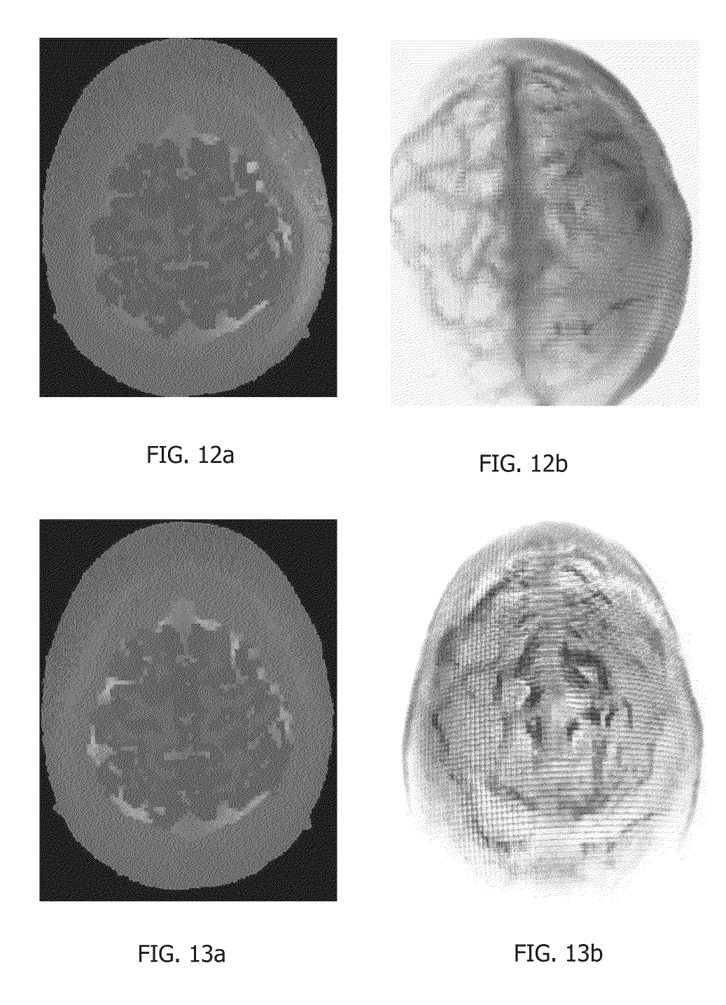

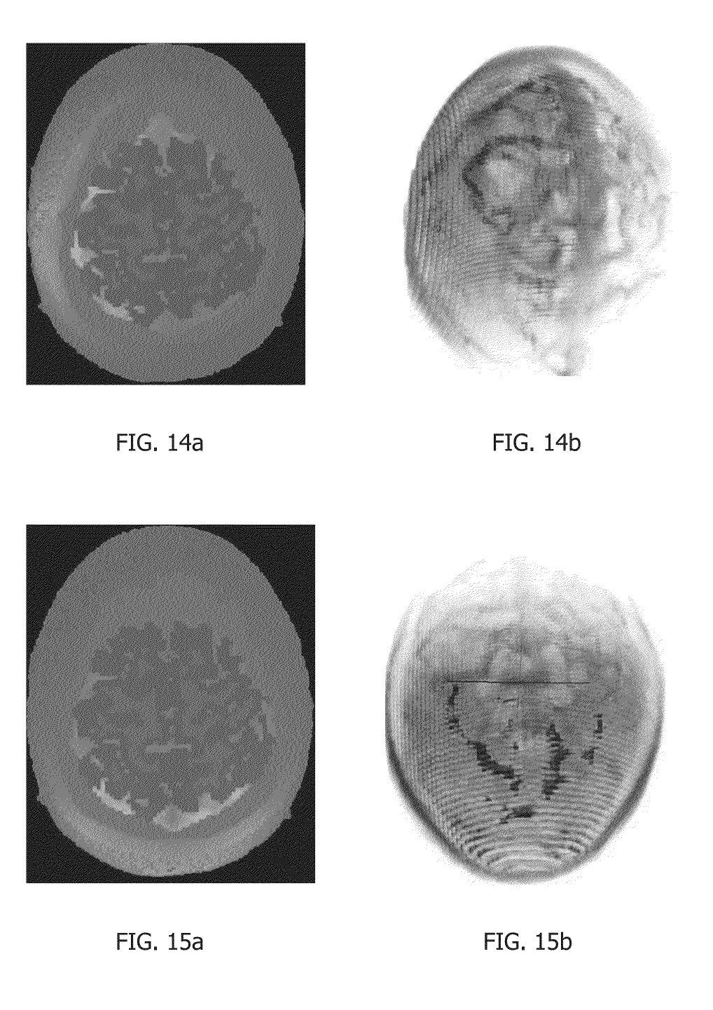

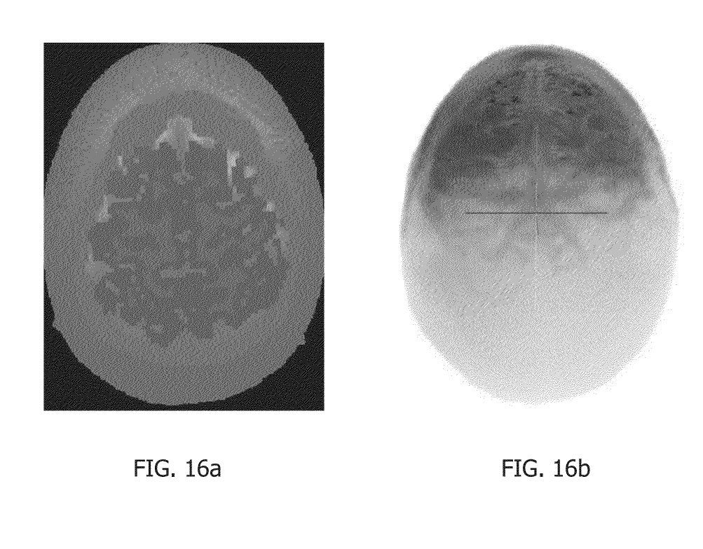

In order to show the capability of the proposed technique for calculating induced current, the MRI data from 25 year old healthy human subject has been used. FIGS. 7(a) and 7(b) show a map of conductivity for a female patient. We have considered the circular coil to be used for stimulation. The coil is placed at the top of the head, as illustrated in FIG. 10. The coil is placed 2 mm away from skin at five different locations as shown in FIG. 11. FIGS. 13 (a)&(b), FIGS. 14 (a)&(b), FIGS. 15 (a)&(b), and FIGS. 16 (a)&(b) show the induced current for locations A, B, C, D, and E, respectively, of the coil in FIG. 11. The run-time of the TMS solver strongly depends on the resolution of the image. As the resolution of the induced current images increases the run-time also increase, because the number of unknown increases.

TABLE II

| Resolution of Image | 4 | mm | 2 | mm |
|---|---|---|---|---|
| Degree of freedom | 373,478 | | 2,395,020 | |
| Number of coil Positions | 61 | | 61 | |
| Total CPU run-time | 01:10:05 | | 13:06:09 | |
| Number of machines | 50 | | 100 | |
| Memory usage | 694,166,384 | kb | 1,109,636,816 | kb |

Table II compares the simulation run-time for 61 positions of the coil for 2 mm and 4 mm image resolutions. The total CPU run-time for 2 mm image resolution using 100 parallel machines took around 13 hours and for 4 mm image resolution the CPU run-time took around 1 hour using 50 parallel machines.

TABLE III

| 1 sample | ≈10:32:00 | |
|---|---|---|
| 2 sample | ≈30 | min |
| 3 sample | ≈7.3 | min |
| 4 sample | ≈2.6 | min |
| 5-61 sample | ≈41 | sec |

Table III shows the run-time for each position (sample) of the coil for 2 mm image resolution. As can be seen in this table, the first sample took around 10.5 hours, second sample around 30 minutes, third sample 7.3 minutes, fourth sample 2.6 minutes, and after that rest of the samples took only 41 seconds to be run. Due to the fact that creating the mesh is a time consuming part of the FEM solver the first sample take a lot of time to be solved. Afterwards the solver run-time decreases rapidly and after the 4th sample, by changing the coil position the eddy current image became ready in just 40 seconds. In one embodiment, in order to provide a real-time TMS solver, it is necessary to let the TMS solver run off-line for at least a few samples before TMS procedure is begun. Further, by increasing the number of computing machines the simulation runtime decreases as shown in Table IV.

TABLE IV

| Resolution of Image | 2 | mm | 2 | mm |
|---|---|---|---|---|
| Number of coil Positions | 61 | | 61 | |
| Number of machines | 100 | | 120 | |
| Memory usage | 1,109,636,816 | kb | 953,054,140 | kb |

| | | |
|---|---|---|
| Total CPU run-time | 13:06:09 | 11:21:11 |

FIGS. 14-16 (a) and (b) show various maps of induced current for circular coils at various positions, A through E, in FIG. 11, i.e., maps of induced current FIGS. 14(a)-16(a) and induced current mapped on the MRI (sagittal plane) FIGS. 14(b)-16(b).

It should be understood that the application is not limited to the details or methodology set forth in the following description or illustrated in the figures. It should also be understood that the phraseology and terminology employed herein is for the purpose of description only and should not be regarded as limiting.

While the exemplary embodiments illustrated in the figures and described herein are presently preferred, it should be understood that these embodiments are offered by way of example only. Accordingly, the present application is not limited to a particular embodiment, but extends to various modifications that nevertheless fall within the scope of the appended claims. The order or sequence of any processes or method steps may be varied or re-sequenced according to alternative embodiments.

It is important to note that the construction and arrangement of the superdirective near field array, or TMS coil array, as shown in the various exemplary embodiments is illustrative only. Although only a few embodiments have been described in detail in this disclosure, those who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements, the position of elements may be reversed or otherwise varied, and the nature or number of discrete elements or positions may be altered or varied. Accordingly, all such modifications are intended to be included within the scope of the present application. The order or sequence of any process or method steps may be varied or re-sequenced according to alternative embodiments. In the claims, any means-plus-function clause is intended to cover the structures described herein as performing the recited function and not only structural equivalents but also equivalent structures. Other substitutions, modifications, changes and omissions may be made in the design, operating conditions and arrangement of the exemplary embodiments without departing from the scope of the present application.

We claim:

1. A superdirective near field array for transcranial magnetic stimulation comprising
a plurality of electromagnetic elements arranged in a superdirective array,
an excitation source for inducing an electrical current in one or more of the electromagnetic elements, and
a controller programmed to position or direct the elements to generate a magnetic field in at least one excitation region of a living organism;
wherein the array coils are configured to focus a predetermined current density in the excitation region or regions using near-field sub-wavelength focusing and to reposition the excitation region to various locations around the head or brain by varying a phase and a magnitude of the electrical current in predetermined array elements.

2. The superdirective near field array of claim 1, further comprising sub-diffraction limit focusing, and superdirective arrays, for focusing electromagnetic fields within the living organism to much smaller dimensions.

3. The superdirective near field array of claim 1, wherein the array generates one or more localized magnetic fields within the living organism and electronically scans at least one structure associated with the living organism.

4. The superdirective near field array of claim 1, wherein the superdirective array is a transcranial magnetic stimulation (TMS) array coil, the TMS array coil configured to focus a maximum current density in a region of the living organism that is a few cubic millimeters.

5. The superdirective near field array of claim 1, wherein the excitation region of the organism may be repositioned within the organism by varying the phase and magnitude of the electrical current in different elements of the superdirective near field array.

6. The superdirective near field array of claim 1, wherein at least one element of the plurality of elements is disposed near an adjacent element, the at least one element and the adjacent element providing electromagnetic coupling therebetween.

7. The superdirective near field array of claim 1, further comprising directivity that is greater than is suggested by a diffraction limit of the elements.

8. The superdirective near field array of claim 1, wherein the plurality of elements comprises a lattice of elements configured to produce one or more null fields at one or more locations apart from a main beam to generate high current magnitudes.

9. The superdirective near field array of claim 1, wherein the electrical currents in adjacent elements of the plurality of elements are nearly 180 degrees out of phase.

10. The superdirective near field array of claim 4, wherein the controller controls electromagnetic coupling and low radiation resistance for the TMS array.

11. The superdirective near field array of claim 10, wherein the TMS array is configured to operate at very low frequencies.

12. The superdirective near field array of claim 4, wherein the TMS array further comprises a shielding material disposed between elements.

13. The superdirective near field array of claim 4, wherein the TMS array further comprises superconducting magnets to achieve the required magnetic field densities.

14. The superdirective near field array of claim 4, wherein the TMS array comprises a 10-element linear array.

15. A method for transcranial magnetic stimulation comprising:
providing a plurality of electromagnetic coils or elements arranged in a superdirective array, an excitation source for inducing an electrical current in one or more of the electromagnetic coils, and a controller programmed to generate a magnetic field;
generating a magnetic field in at least one excitation region of a living organism;
varying a phase or a magnitude of the electrical current in predetermined array elements; and
repositioning the excitation region to one or more locations around the living organism.

16. The method of claim 15, wherein generating a magnetic field further comprises focusing a predetermined current density in the at least one excitation region.

17. The method of claim 15, the method further comprising:
identifying phasing methods for an arbitrary set of sources to provide the smallest focal spot for currents excited in the brain.

18. The method of claim 15, wherein generating a magnetic field comprises exciting elements of the array such that a maximum field intensity is the same for each element of the respective elements and wherein the size of a focused region of maximum current density is a few cubic millimeters.

19. The method of claim 15, further comprising varying the phase and the magnitude of the electrical current in elements of the array and moving the focused region about the living organism.

20. The method of claim 15, further comprising wherein a size and a shape of an excited region is varied to excite broad regions of the organism or entirely separate regions of the organism simultaneously by applying different waveforms to the array elements.
